(19) United States
(12) Patent Application Publication
(10) Pub. No.: US 2012/0116322 A1
(43) Pub. Date: May 10, 2012

(54) METHOD TO FABRICATE A NEEDLE HAVING A TAPERED PORTION BETWEEN A DISTAL TIP AND A LONGITUDINAL CHANNEL

(75) Inventors: Damon Douglas Brink, Ventura, CA (US); James Allen Reitz, Ventura, CA (US); Sandra Gayle Reid, Goleta, CA (US)

(73) Assignee: INTRIMED TECHNOLOGIES, INC., OXNARD, CA (US)

(21) Appl. No.: 12/941,046

(22) Filed: Nov. 6, 2010

(51) Int. Cl. A61M 25/00; B21D 51/16

(52) U.S. Cl. 604/264; 72/324

(57) ABSTRACT

A novel method to fabricate a needle includes stamping an elongated shape in a flat metal sheet. The elongated shape defines a longitudinal axis and two edges that are substantially parallel to the longitudinal axis. Each of the two edges includes an inward curve with relative alignment to create a relatively narrow neck. A longitudinal channel is formed to give the elongated shape a U-shaped cross-section in a plane normal to the longitudinal axis. The method includes punching through the neck with a punch that slides along a punching axis that intersects the longitudinal axis with an angle in the range 15° to 50°. Alternatively the method includes coining a thinned region that spans the neck and may define a V-shape in a major plane of the flat metal sheet, and separating the elongated shape along the thinned region after coining.

### FIELD OF THE INVENTION

The present invention relates generally to needles for insertion into human and animal tissues, and in particular to methods to fabricate such needles.

### BACKGROUND

Various types of hollow or channeled needles are used to inject medicines or other liquids into human or animal vessels or tissues, and/or to remove or sample fluids from such vessels or tissues, and/or to sample the tissues themselves (e.g. biopsy needles), and/or to insert or guide lumens or other medical device projections, tubes, or sensors into the vessels or tissues. For example, hollow or channeled needles have been used to introduce slender tubes into human vessels or tissues to facilitate the testing of glucose in the blood of patients (e.g. diabetic patients).

The tips of such needles must be adequately sharp, with a tip contour that facilitates puncture and entry through human skin with an acceptably low insertion force. Otherwise, an insertion force that is higher than desirable may result in unacceptable pain during insertion. However, grinding a sharp point on the end of the needle to create a needle tip requires a large proportion of the of the overall fabrication time, and so grinding can be an undesirably expensive step in the needle manufacturing process.

Hence, there is a need in the art for methods and designs that can enable faster and/or less expensive fabrication of hollow or channeled needles with a sharp tip.

### SUMMARY

A novel method to fabricate a needle is disclosed. An elongated shape is stamped in a flat metal sheet. The elongated shape has a body portion and a distal portion. The elongated shape defines a longitudinal axis and two edges that are substantially parallel to the longitudinal axis in the body portion. Each of the two edges includes an inward curve in the distal portion. Each inward curve is aligned relative to the other so as to create a neck in the distal portion. The neck is narrower than a width between the two edges in the body portion. A longitudinal channel is formed in the elongated shape. The longitudinal channel is aligned with the longitudinal axis and gives the elongated shape a U-shaped cross-section in a plane normal to the longitudinal axis at the body portion. The method includes punching through the neck with a punch that slides along a punching axis that intersects the longitudinal axis with an angle in the range 15° to 50°. Alternatively the method includes coining a thinned region that spans the neck and may define a V-shape in a major plane of the flat metal sheet, and separating the distal portion along the thinned region after coining.

### BRIEF DESCRIPTION OF THE DRAWINGS

FIG. 1 is a plan view of a plurality of elongated shapes being stamped from a flat sheet according to an embodiment of the present invention.

FIG. 2 is an expanded view of the stamped sheet of FIG. 1.

FIG. 3A is a plan view of an elongated shape before its neck is punched according to an embodiment of the present invention.

FIG. 3B is a cross-sectional view of the elongated shape of FIG. 3A.

FIG. 4 is a plan view of a distal portion of an elongated shape according to an embodiment of the present invention.

FIG. 5A is a side perspective view of a punching apparatus according to an embodiment of the present invention.

FIG. 5B is an expanded view of the cutting portion of the punching apparatus of FIG. 5A.

FIG. 6A is a plan view of a needle according to an embodiment of the present invention.

FIG. 6B is an enlarged perspective view of the tapered tip portion of the needle of FIG. 6A.

FIG. 6C is an enlarged top view of the tapered tip portion of the needle of FIG. 6A.

FIG. 7 is a perspective view of the tapered tip portion of a needle according to another embodiment of the present invention.

FIG. 8A is a perspective view of the tapered tip portion of a needle according to another embodiment of the present invention.

FIG. 8B is a side profile of the tapered tip portion of the needle of FIG. 8A.

FIG. 9 is a perspective view of the tapered tip portion of a needle according to another embodiment of the present invention.

FIG. 10A is a perspective view of a double needle according to an embodiment of the present invention.

FIG. 10B is a perspective view of a double needle according to another embodiment of the present invention.

FIG. 10C is a perspective view of a double needle according to another embodiment of the present invention.

### DESCRIPTION OF THE PREFERRED EMBODIMENTS

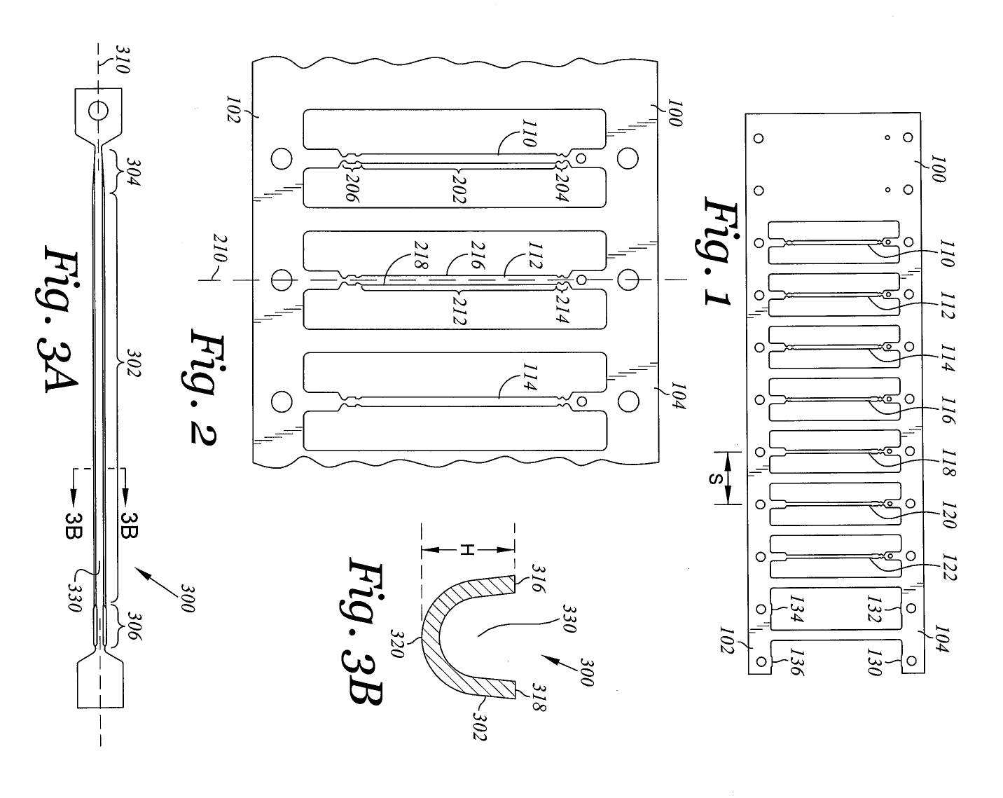

A novel method to fabricate a needle is disclosed herein. FIG. 1 is a plan view of a plurality of elongated shapes 110, 112, 114, 116, 118, 120, and 122 being stamped from a flat metal sheet 100 according to an embodiment of the present invention. The metal sheet 100 may comprise stainless steel, for example. The stamping may define a first metal strip 102, and/or a second metal strip 104 in the metal sheet 100 that is substantially parallel to the first metal strip 102. Each of the plurality of elongated shapes 110, 112, 114, 116, 118, 120, and 122 optionally may be spaced from another by a constant inter-needle interval spacing S, for example to simplify indexing if the metal sheet 100 is fed into a stamping tool during manufacture.

Alternatively, in certain embodiments the plurality of elongated shapes 110, 112, 114, 116, 118, 120, and 122 may optionally be grouped in pairs, with the two elongated shapes of each pair being spaced from each other by an intra-dual-needle spacing that is less than an inter-pair spacing. In certain embodiments, the two elongated shapes of each pair are never further separated during or after manufacture, but rather remain interconnected by a metal bridge, which may comprise the first metal strip 102, or alternatively may be in addition to and not a portion of the first or second metal strips 102, 104. In certain alternative embodiments, the bridge between the elongated shapes of each pair is not a metal bridge (e.g. a plastic overmold). Where the first metal strip 102 is used as the metal bridge for a dual needle pair, the first metal strip 102 may be crimped to reduce the spacing between the two elongated shapes of a pair, for example, from initially being the greater inter-pair spacing (before crimping) to ultimately being the lesser intra-dual-needle spacing (after crimping). Even in embodiments where the metal bridge is not the first metal strip 102, but rather the metal bridge is a dedicated and distinct sub-structure between the body portions of adjacent elongated shapes, such metal bridge may be crimped to allow reduced intra-dual-needle spacing despite process limitations associated with forming channels in adjacent elongated shapes.

FIG. 2 is an expanded view of the stamped metal sheet 100 of FIG. 1. The elongated shape 110 has a body portion 202 and a distal portion 204. The elongated shape 110 further comprises a root portion 206, with the body portion 202 being disposed between the root portion 206 and the distal portion 204. The elongated shape 112 defines a longitudinal axis 210 and two edges 216, 218 that are substantially parallel to the longitudinal axis 210 in the body portion 212. Each of the two edges 216, 218 includes an inward curve in the distal portion 214. Each inward curve is aligned relative to the other so as to create a neck in the distal portion 214. The neck is narrower than a width between the two edges 216, 218 in the body portion 212.

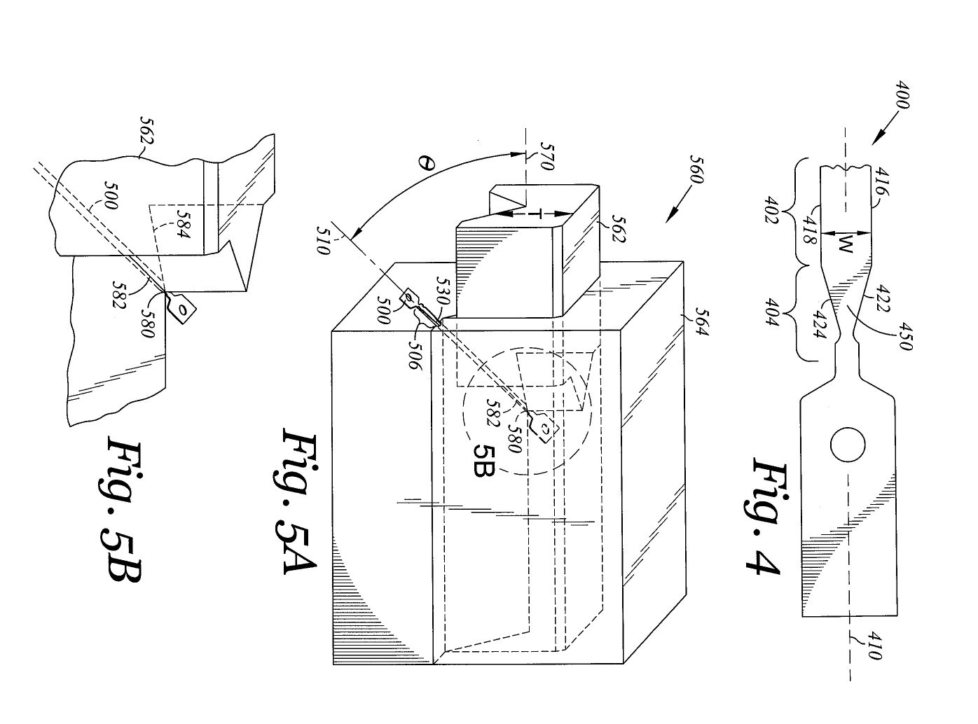

Each elongated shape 110, 112, 114, 116, 118, 120, 122 may be sheared from the metal sheet 100. For example, FIG. 1 depicts locations 130 and 132 where longitudinal shapes were sheared off from the metal sheet 100 (proximate to their distal portions), and locations 134 and 136 where longitudinal shapes were sheared off from the metal sheet 100 (proximate to their root portions). FIG. 3A is a plan view of an elongated shape 300 having a body portion 302, a root portion 306, and a distal portion 304, according to an embodiment of the present invention. The elongated shape 300 of FIG. 3A is shown after being sheared from the metal sheet 100 (and after a forming operation to create a longitudinal channel 330), but before a neck in its distal portion 304 is punched.

FIG. 3B is a cross-sectional view of the elongated shape 300 in its body portion 302, which more clearly shows the shape of the longitudinal channel that has been formed in the elongated shape 300. Now referring additionally to FIG. 3B, the longitudinal channel 330 is aligned with the longitudinal axis 310 and may give the elongated shape 300 a U-shaped cross-section in a plane normal to the longitudinal axis 310 at the body portion 302. Forming of the channel may be accomplished by multiple forming steps, each making the channel 330 deeper and the U-shape of the cross-section more pronounced. In the embodiment of FIGS. 3A-3B, the U-shaped cross section defines a U bottom 320 and two U tops 316 and 318, and the U-shaped cross-section defines a height H that is measured from the U bottom 320 to either of the U tops 316 or 318.

If the channel forming is optionally continued to bring the U tops 316, 318 of the U shaped cross-section towards each other to partially or completely close the top of the channel 330, then the forming tool may ultimately need to be removed from the channel 330 longitudinally (i.e. by relative translation along the longitudinal axis 310), after forming the longitudinal channel 330 in the elongated shape 300. In this case, the channel 330 in the body portion 302 may ultimately have a substantially closed hollow cross-section with a top seam that is parallel to the longitudinal axis 310, rather than having an open U-shaped cross section in the body portion 302. In this case, although the substantially closed channel in the body portion 302 ultimately may have a cross-section that looks more like the letter “O,” for convenience it will still be referred to as a “U-shaped” cross section herein. Also, in the case that the channel forming is continued to bring the tops 316, 318 of the U-shaped cross section together and thereby substantially close the top of the channel 330, the U tops 316, 318 may be optionally welded together to better close the top seam.

In certain embodiments, the height H of the U-shaped cross section is greater in the body portion 302 than in the distal portion 304, because the width of the elongated shape 300 was less in the distal portion 304 before the longitudinal channel 330 was formed. For example, the distal portion 304 of the elongated shape 300 may have included a neck that was narrower than a width between the two edges 316, 318 in the body portion 302, prior to the longitudinal channel 330 being formed. Preferably, the U-shaped cross section defines a greater height H in the body portion 302 and a lesser height in the distal portion 304 where the neck is later punched. For example, in certain embodiments, the lesser height may preferably be 10% to 55% of the greater height. Also for example, in certain embodiments the lesser height may be in the range 25 to 800 microns. In certain embodiments these inequalities may reduce the likelihood of the U-shaped cross section undesirably folding upon itself in the distal portion 304 in response to punching, and also allow an acceptable lifetime of the punch for practical commercial manufacturing of the needles.

FIG. 4 is a plan view of a distal portion 404 of an elongated shape 400 according to an embodiment of the present invention (shown before channel forming). The elongated shape 400 defines a longitudinal axis 410 and two edges 416, 418 that are substantially parallel to the longitudinal axis 410 in the body portion 402. Each of the two edges 416, 418 includes an inward curve 422, 424 in the distal portion 404. Each inward curve 422, 424 is aligned relative to the other so as to create a neck 450 in the distal portion 404. The neck 450 is narrower than a width W between the two edges 416, 418 in the body portion 402. Note that in the embodiment of FIG. 4, the neck 450 is hour glass shaped because, from left to right in FIG. 4, it gets narrower and then wider again. That is, the neck 450 is hour glass shaped because it includes a relatively narrow portion between two relatively wider portions.

In certain embodiments the neck 450 may be punched after a longitudinal channel is formed in the elongated shape 400, preferably with a punch that slides along a punching axis that intersects the longitudinal axis 410 with an angle (tilted in or out of the page in FIG. 4) in the range 15° to 50°. Although punching through the neck 450 is preferably accomplished subsequently to forming the longitudinal channel in the elongated shape 400, it need not be accomplished immediately subsequently; there could be intermediate steps.

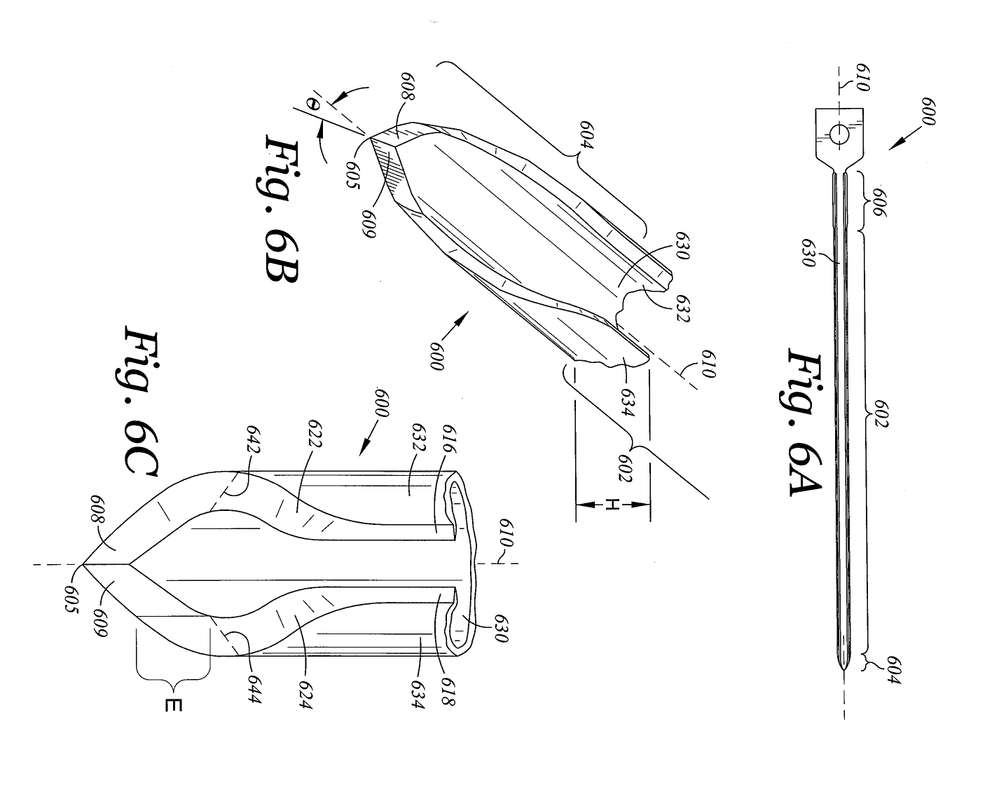

For example, FIG. 5A is a side perspective view of an example punching apparatus 560, according to an embodiment of the present invention. The punching apparatus 560 may include a moving punch 562 and a stationary receiver 564. The moving punch 562 may slide with respect to the stationary receiver 564, along a punching axis 570. An elongated shape 500, having a root portion 506 and a longitudinal channel 530, may be held by the stationary receiver 564 while the moving punch 562 cuts through a neck of the distal portion of the elongated shape 500. An angle 0 between the punching axis 570 and the longitudinal axis 510 of the elongated shape 500 (while it is held by the stationary receiver 564), is preferably in the range 15° to 50°.

FIG. 5B is an expanded view of the cutting portion of the punching apparatus of FIG. 5A. In the embodiment of FIGS. 5A-5B, the punch 562 may include a leading point 580 that is aligned with the punching axis 570 and that cuts through the neck of a distal portion of the elongated shape 500 (at an intersection with its longitudinal axis 510), during punching. The punch 562 has a leading V-shaped edge that has first and second wings 582, 584 that extend from the leading point 580. In the embodiment of FIG. 5A, the punch 562 also defines a punch thickness T that may be tapered along each of the first and second wings 582, 584, for example so that it is least at the punching axis 570 and preferably increases away from the punching axis 570.

In this regard, the term “V-shaped” does not require that the first and second wings 582, 584 be straight edged (as shown in FIG. 5B). Rather, the first and second wings 582, 584 could alternatively be curved. For example, the first and second wings 582, 584 could be curved to retard the timing of the engagement of the edges further from the centerline (football nose shape) or else to advance the timing of the engagement of the edge further from the centerline (gull wing shape). In certain embodiments, the wing edges preferably engage the material of the needle everywhere simultaneously.

In the embodiment of FIGS. 5A-5B during punching, the first wing 582 cuts through the neck of the distal portion of the elongated shape 500 on a first side of its longitudinal axis 510, and the second wing 584 cuts through the neck of the distal portion of the elongated shape 500 on a second side of its longitudinal axis 510 that is opposite the first side. In this way, the first wing 582 creates a first cut facet through the neck of the distal portion of the elongated shape 500 on the first side of the longitudinal axis 510, and the second wing 584 creates a second cut facet through the neck of the distal portion of the elongated shape 500 on the second side of the longitudinal axis 510.

In certain embodiments, the leading V-shaped edge of the punch 562 may be duplicated in the punching apparatus 560, so that punching through the neck in the distal portion of one elongated shape may be accomplished simultaneously with punching through a plurality of other similar distal portions of a plurality of other similar elongated shapes. In such embodiments, the root portions of the elongated shapes may be connected together (e.g. by a first metal strip like the first metal strip 102 of FIGS. 1-2) to facilitate handling and positioning of the elongated shapes in the punching apparatus. Prior to punching, the distal portions of the elongated shapes may also be initially connected together (e.g. by a second metal strip like the second metal strip 104 of FIGS. 1-2) to also help facilitate handling and positioning of the elongated shapes in the punching apparatus. Alternatively, the root portions of the elongated shapes may be connected together (e.g. by a first metal strip like the first metal strip 102 of FIGS. 1-2) without the presence of a second metal strip.

Alternatively, needles according to the present invention may be fabricated from elongated shapes without punching through a neck of the distal portion of each elongated shape. For example, and now referring again to FIG. 4, a thinned region that spans the neck 450 of the distal portion 404 of the elongated shape 400 may first be coined, and then the distal portion 404 may be separated along the thinned region after coining. In certain embodiments, the thinned region may define a V-shape in a major plane of the flat metal sheet (e.g. flat metal shape 100 of FIGS. 1-2). In certain embodiments, a longitudinal channel may preferably be formed in the elongated shape 400 after coining the thinned region that spans the neck 450, though this order of steps is not necessary. In certain embodiments, such a longitudinal channel may preferably be formed in the elongated shape 400 after the distal portion 404 is separated along the coined thinned region, though this order of steps is not necessary.

FIG. 6A is a plan view of a needle 600 according to an embodiment of the present invention, that may have been punched from an elongated shape by the use (described in previous paragraphs) of the punching apparatus 560. The needle 600 has a tapered portion 604, a root portion 606, and a body portion 602 between the tapered portion 604 and the root portion 606. FIG. 6B is an enlarged perspective view of the tapered portion 604 of the needle 600. The body portion 602 of the needle 600 includes a longitudinal channel 630 defining a longitudinal axis 610.

In the embodiment of FIGS. 6A-6B, the tapered portion 604 includes a distal tip 605, with the tapered portion 604 being disposed between the distal tip 605 and the body portion 602. The tapered portion 604 may have a U-shaped cross-section in a plane normal to the longitudinal axis 610, the U-shaped cross-section defining an open top and a closed bottom. This may be true even if the body portion 602 has a closed cross-section with a closed top and closed bottom.

As shown in FIG. 6B, the distal tip 605 has a cut facet 608 that is inclined with respect to the longitudinal axis 610 by an angle θ. In certain embodiments the angle θ is in the range 15 degrees to 50 degrees. In the embodiment of FIG. 6B, the distal tip 605 also includes a cut facet 609 that is likewise inclined with respect to the longitudinal axis 610. Also, in the embodiment of FIG. 6B, a plane that is parallel to and tangent the cut facet 608 does not intersect any part of the needle 600 (except the cut facet 608). Such non-intersection may help facilitate the punching process by providing tool clearance from needle features that would fold rather than shear, and thereby may improve the manufacturability of the needle 600. In the embodiment of FIG. 6B, the longitudinal channel 630 is open at the top and has channel side walls 632, 634 that define a wall height H (measured normal to the longitudinal axis with the closed bottom used as a datum) that is preferably but not necessarily in the range 0.1 mm to 1.5 mm. In this context, the meaning of datum is the location from which a dimension is measured.

FIG. 6C is an enlarged top view of the tapered tip portion of the needle of FIG. 6A. Now referring additionally to FIG. 6C, the first and second cut facets 608, 609 in the needle 600 may result from material shear during punching. Each of the first and second cut facets 608, 609 is preferably adjacent and joining the inward curve 622, 624 of a respective one of the two edges 616, 618 of the needle 600 in its distal portion 604. Specifically, in the embodiment of FIG. 6C, the first and second cut facets 608, 609 join the inward curves 622, 624 at the facet boundaries 642, 644, respectively. Preferably, any angular edge discontinuity (in or out of the page in FIG. 6C) at the facet boundaries 642, 644—where the first and second cut facets 608, 609 join the inward curves 622, 624 (of the neck in the distal portion of the elongated shape from which needle 600 was punched)—is no more than 5°. Ultimately, this dimensional inequality may help desirably reduce or limit the insertion force of the manufactured needles.

Note also that FIG. 6C depicts an effective material thickness E that is experienced by the cutting portion of the punch as it attempts to shear the needle tip. This effective material thickness E is related to the angle 0 between the longitudinal axis 610 of the needle and the punching axis (e.g. punching axis 570 as shown in FIG. 5A). For example, the minimum effective material thickness Eₘᵢₙ is equal to t/(sin θ), where the symbol t represents the thickness of the metal sheet (e.g. flat metal sheet 100 of FIGS. 1-2) from which the needles are manufactured. On the other hand, the maximum possible effective material thickness Eₘₐₓ is equal to Hₘₐₓ/(sin θ), where Hₘₐₓ is the maximum wall height of the channel side walls 632, 634. Practically, it may not be desirable for the effective material thickness E to be Eₘₐₓ, since punching may then fold or partially locally collapse the channel side walls 632, 634 and/or tool life may be reduced. For example, in certain embodiments, E≈2t/(sin θ) may be preferred. In certain embodiments, the material may have a metal sheet thickness in the range 0.012 mm to 0.13 mm, and an effective material thickness E in the range 0.15 mm to 1 mm.

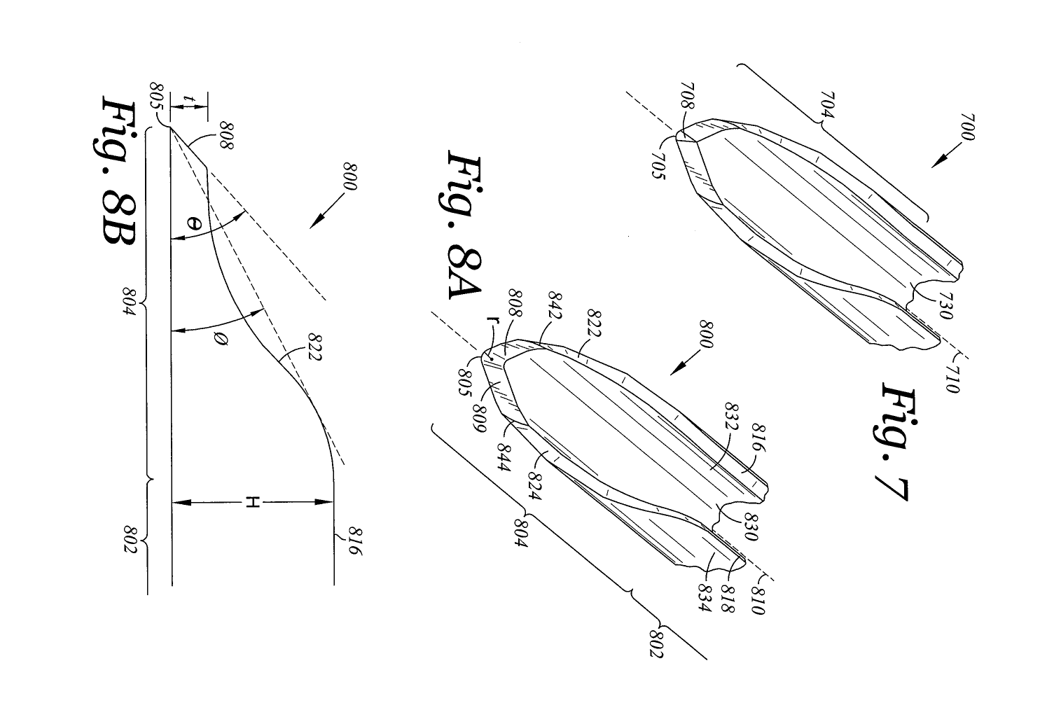

FIG. 7 is a perspective view of a tapered tip portion 704 of a needle 700 according to another embodiment of the present invention that may have been punched from an elongated shape by the use (described in previous paragraphs) of the punching apparatus 560. The tapered portion 704 of the needle 700 includes a distal tip 705. The needle 700 also includes a longitudinal channel 730 defining a longitudinal axis 710. In the embodiment of FIG. 7, the tapered portion 704 may have a U-shaped cross-section in a plane normal to the longitudinal axis 710, the U-shaped cross-section defining an open top and a closed bottom. This may be true even if a body portion of the needle 700 has a closed cross-section with a closed top and closed bottom.

As shown in FIG. 7, the distal tip 705 has a cut facet 708 that is inclined with respect to the longitudinal axis 710, and that may result from material shear during punching. Also, in the embodiment of FIG. 7, a plane that is parallel to and tangent the cut facet 708 does not intersect any part of the needle 700 (except the cut facet 708). Such non-intersection may help facilitate the punching process by providing tool clearance from needle features that would fold rather than shear, and thereby may improve the manufacturability of the needle 700. In the embodiment of FIG. 7, the distal tip 705 may define a distal tip radius of curvature in a plane that is parallel with the longitudinal axis 710 (and tangent to the closed bottom). In the case that the distal tip 705 is created by a so-called “split-punch,” which is a punch that has two cutting edges that meet at a material seam in the punching tool, the distal tip radius of curvature may preferably be in a lower portion of the range 12 microns to 125 microns.

FIG. 8A is a perspective view of a needle 800 according to another embodiment of the present invention, that may have been punched from an elongated shape by the use (described in previous paragraphs) of the punching apparatus 560. The needle 800 has a tapered portion 804 that includes its distal tip 805, and a body portion 802. FIG. 8B is a side profile of the tapered tip portion of the needle 800. The body portion 802 of the needle 800 includes a longitudinal channel 830 defining a longitudinal axis 810. In the embodiment of FIGS. 8A-8B, the tapered portion 804 is disposed between the distal tip 805 and the body portion 802. The tapered portion 804 may have a U-shaped cross-section in a plane normal to the longitudinal axis 810, the U-shaped cross-section defining an open top and a closed bottom. This may be true even if the body portion 802 has a closed cross-section with a closed top and closed bottom.

As shown in FIGS. 8A-8B, the distal tip 805 has a cut facet 808 that is inclined with respect to the longitudinal axis 810 by an angle θ. In certain embodiments, the angle θ is preferably in the range 15 degrees to 50 degrees. In the embodiment of FIGS. 8A-8B, the distal tip 805 also includes a cut facet 809 that is likewise inclined with respect to the longitudinal axis 810. Also, in the embodiment of FIGS. 8A-8B, a plane that is parallel to and tangent the cut facet 808 does not intersect any part of the needle 800 (except the cut facet 808). This can be seen in FIG. 8B since the angle 0 of the cut facet 808 is less than the angle φ to the nearest point of intersection with the rest of the needle 800. Such non-intersection may help facilitate the punching process by providing tool clearance from needle features that would fold rather than shear, and thereby may improve the manufacturability of the needle 800.

In the embodiment of FIGS. 8A-8B, the longitudinal channel 830 is open at the top and has channel side walls 832, 834 that define a wall height H (measured normal to the longitudinal axis with the closed bottom used as a datum) that is preferably but not necessarily in the range 0.1 mm to 1.5 mm. The first and second cut facets 808, 809 in the needle 800 may result from material shear during punching. Note that, in FIG. 8B, the symbol t represents the thickness of the metal sheet (e.g. flat metal sheet 100 of FIGS. 1-2) from which the needle 800 was manufactured.

In the embodiment of FIGS. 8A-8B, each of the first and second cut facets 808, 809 is preferably adjacent and joining the inward curve 822, 824 of a respective one of the two edges 816, 818 of the needle 800 in its distal portion 804. Specifically, in the embodiment of FIGS. 8A-8B, the first and second cut facets 808, 809 join the inward curves 822, 824 at the facet boundaries 842, 844, respectively. Preferably, any angular edge discontinuity at the facet boundaries 842, 844—where the first and second cut facets 808, 809 join the inward curves 822, 824 (of the neck in the distal portion of the elongated shape from which needle 800 was punched)—is no more than 5°. Ultimately, this dimensional inequality may help desirably reduce or limit the insertion force of the manufactured needles.

In the embodiment of FIG. 8A, the distal tip may define a distal tip radius of curvature (labeled r in FIG. 8A) in a plane that is parallel with the longitudinal axis 810 and tangent to the closed bottom. In the case that the distal tip 805 is created by a punch having two cutting edges that meet at an interior corner of a single piece punching tool (where the interior corner does not correspond to any seam or material discontinuity, and so is characterized by an interior fillet), the distal tip radius of curvature may preferably be in a middle or upper portion of the range 12 microns to 125 microns.

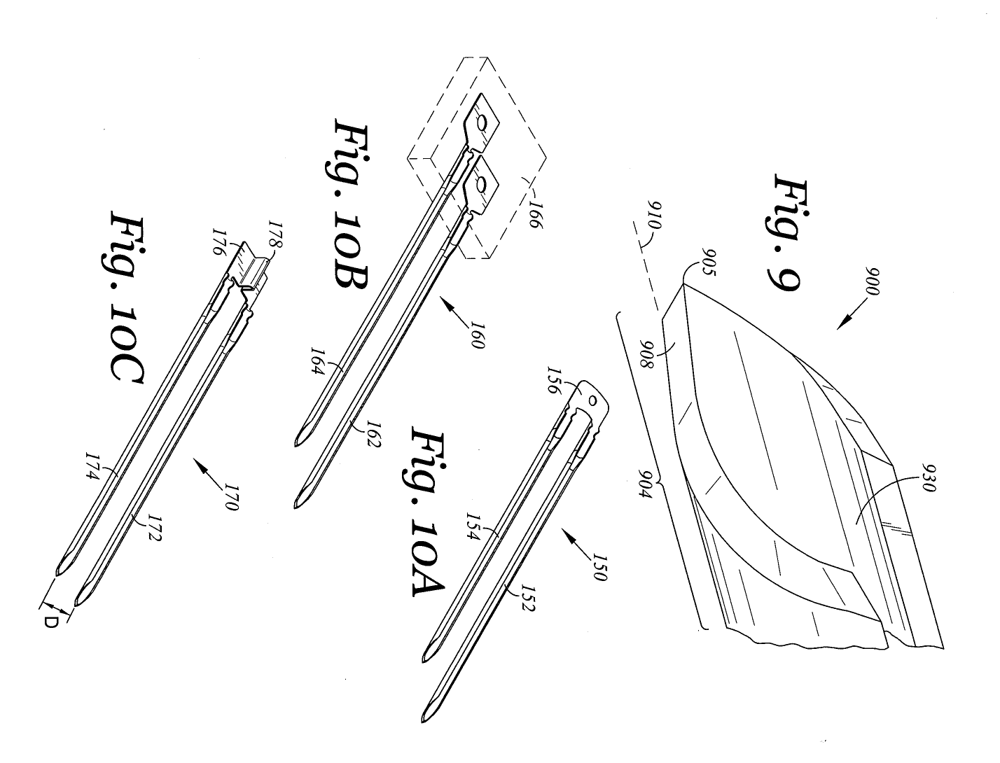

FIG. 9 is a perspective view of a tapered tip portion 904 of a needle 900 according to another embodiment of the present invention. The tapered portion 904 of the needle 900 includes a distal tip 905. The distal tip 905 may be punched from an elongated shape by the aforedescribed use of a punching apparatus like punching apparatus 560, except with a reversed relative angle between the elongated shape longitudinal axis with respect to the punching axis (and a modified leading edge for the punch 562). In such embodiment, the punching axis would still preferably (but not necessarily) intersect the longitudinal axis with an angle in the range 15° to 50°, though that relative angle be reversed.

The needle 900 also includes a longitudinal channel 930 defining a longitudinal axis 910. In the embodiment of FIG. 9, punching the distal tip 905 to create a reverse bevel preferably (but not necessarily) precedes the forming of the longitudinal channel 930. In the embodiment of FIG. 9, the tapered portion 904 may have a U-shaped cross-section in a plane normal to the longitudinal axis 910, the U-shaped cross-section defining an open top and a closed bottom. This may be true even if a body portion of the needle 900 has a closed cross-section with a closed top and closed bottom.

As shown in FIG. 9, the distal tip 905 has a cut facet 908 (that may result from material shear during punching) having a reverse bevel with respect to the longitudinal axis 910. In this context, the cut facet 908 is said (and shown) to have a “reverse bevel” since its orientation causes the most protruding tip 905 of the needle 900 to be at the inner surface of the needle 900 rather than the outer surface of the needle 900. Such a reverse bevel of the cut facet 908 may be advantageous for use of the needle 900 as a biopsy needle to collect small tissue samples.

FIGS. 10A, 10B, and 10C are perspective views of double needles 150, 160, and 170 according to certain other embodiments of the present invention, that may have been punched from elongated shapes by the use (described in previous paragraphs) of the punching apparatus 560, or may have been fabricated from elongated shapes by coining and separating. In the embodiment of FIG. 10A, the double needle 150 includes a pair of needles 152 and 154 that are connected by a metal bridge 156. The bridge 156 holds needles 152 and 154 so that their tips are both laterally and longitudinally offset. In the embodiment of FIG. 10B, the double needle 160 includes a pair of needles 162 and 164 that are connected by a non-metal bridge (e.g. plastic overmold 166 over the root ends of needles 162 and 164). In the embodiment of FIG. 10B, the bridge (plastic overmold 166) optionally holds needles 162 and 164 so that their tips are both laterally and longitudinally offset.

In the embodiment of FIG. 10C, the double needle 170 includes a pair of needles 172 and 174 that are connected by a portion 176 of a first metal strip (like the first metal strip 102 of FIGS. 1-2) so that their tips are laterally but not longitudinally offset. The metal strip portion 176 includes a crimp 178 that reduces the spacing D between the pair of needles 172, 174 so that it is less than it would be without the crimp 178. In certain embodiments, but for the crimp 178 the spacing between the pair of needles 172, 174 would be the same as the single needle spacing S that is shown in FIG. 1.

In the foregoing specification, the invention is described with reference to specific exemplary embodiments, but those skilled in the art will recognize that the invention is not limited to those. It is contemplated that various features and aspects of the invention may be used individually or jointly and possibly in a different environment or application. The specification and drawings are, accordingly, to be regarded as illustrative and exemplary rather than restrictive. For example, the word “preferably,” and the phrase “preferably but not necessarily,” are used synonymously herein to consistently include the meaning of “not necessarily” or optionally. “Comprising,” “including,” and “having,” are intended to be open-ended terms.

We claim:

1. A method to fabricate a needle, comprising:
stamping an elongated shape in a flat metal sheet, the elongated shape having a body portion and a distal portion, the elongated shape defining a longitudinal axis and two edges that are substantially parallel to the longitudinal axis in the body portion, each of the two edges including an inward curve in the distal portion, each inward curve being aligned relative to the other so as to create a neck in the distal portion, the neck being narrower than a width between the two edges in the body portion;
forming a longitudinal channel in the elongated shape, the longitudinal channel being aligned with the longitudinal axis, the longitudinal channel giving the elongated shape a U-shaped cross-section in a plane normal to the longitudinal axis at the body portion;
punching through the neck with a punch that slides along a punching axis that intersects the longitudinal axis with an angle in the range 15° to 50°.

2. The method of claim 1 wherein the punch includes a leading point that is aligned with the punching axis and that cuts through the neck at the longitudinal axis during punching.

3. The method of claim 2 wherein the punch has a leading V-shaped edge that has first and second wings that extend from the leading point, and wherein during punching the first wing cuts through the neck on a first side of the longitudinal axis and the second wing cuts through the neck on a second side of the longitudinal axis that is opposite the first side.

4. The method of claim 3 wherein the U-shaped cross section defines a U bottom and two U tops, the U-shaped cross-section defining a height that is measured from the U bottom to either of the U tops, and wherein the U-shaped cross section defines a greater height in the body portion and a lesser height where the first and second wings cut through the neck.

5. The method of claim 4 wherein the lesser height is 10% to 55% of the greater height.

6. The method of claim 5 wherein the lesser height is in the range 25 to 800 microns.

7. The method of claim 4, further comprising another forming of the longitudinal channel to bring the U tops together in the body portion such that the U-shape becomes a substantially closed hollow shape in the body portion, the substantially closed hollow shape having a top seam that is parallel to the longitudinal axis.

8. The method of claim 7 wherein the lesser height is 10% to 55% of the greater height.

9. The method of claim 8 further comprising welding the U tops together at the top seam so that the top seam is closed.

10. The method of claim 3 wherein the punch defines a punch thickness, and wherein the punch thickness is tapered along each of the first and second wings so that the punch thickness is least at the punching axis and increases away from the punching axis.

11. The method of claim 3 wherein the first wing creates a first cut facet through the neck on the first side of the longitudinal axis, and the second wing creates a second cut facet through the neck on the second side of the longitudinal axis, each of the first and second cut facets being adjacent and joining the inward curve of a respective one of the two edges of the elongated shape in the distal portion.

12. The method of claim 11 wherein the neck is hour-glass shaped before the punch punches through it, and wherein there is no angular edge discontinuity greater than 5° where the first cut facet joins the inward curve of the respective one of the two edges.

13. The method of claim 11 wherein the neck is hour-glass shaped before the punch punches through it, and wherein there is no angular edge discontinuity greater than 5° where the second cut facet joins the inward curve of the respective one of the two edges.

14. The method of claim 1 further comprising removing a metal forming tool from the elongated shape by relative translation along the longitudinal axis, after forming the longitudinal channel in the elongated shape.

15. The method of claim 1 wherein punching through the neck is subsequent to forming the longitudinal channel in the elongated shape.

16. The method of claim 1 wherein the elongated shape further comprises a root portion, with the body portion being disposed between the root portion and the distal portion, and wherein punching through the neck in the distal portion of the elongated shape is accomplished simultaneously with punching through a plurality of other similar distal portions of a plurality of other similar elongated shapes, the root portion of the elongated shape being connected to the plurality of other similar elongated shapes by a first metal strip.

17. The method of claim 16 wherein the distal portion of the elongated shape is connected to the other similar distal portions of the plurality of other similar elongated shapes by a second metal strip.

18. The method of claim 17 wherein the second metal strip is substantially parallel to the first metal strip.

19. The method of claim 16 wherein each of the plurality of other similar elongated shapes is spaced from another by a first inter-needle interval spacing.

20. The method of claim 16 wherein the plurality of other similar elongated shapes includes a plurality of pairs of other similar elongated shapes, the other similar elongated shapes of each pair being interconnected by a bridge, and wherein each pair includes two of the other similar elongated shapes that are spaced from each other by an intra-dual-needle spacing, and wherein each pair is spaced from another pair by an inter-pair spacing.

21. The method of claim 20 wherein the inter-pair spacing is greater than the intra-dual-needle spacing.

22. The method of claim 21 wherein the bridge comprises a portion of the first metal strip.

23. The method of claim 21 wherein the bridge comprises a crimp.

24. The method of claim 20 wherein the inter-pair spacing is equal to the intra-dual-needle spacing.

25. The method of claim 20 wherein the inter-pair spacing is less than the intra-dual-needle spacing.

26. The method of claim 20 wherein the bridge comprises a plastic overmold.

27. A method to fabricate a needle, comprising:
stamping an elongated shape in a flat metal sheet, the flat metal sheet defining a major plane, the elongated shape having a body portion and a distal portion, the elongated shape defining a longitudinal axis and two edges that are substantially parallel to the longitudinal axis in the body portion, each of the two edges including an inward curve in the distal portion, each inward curve being aligned relative to the other so as to create a neck in the distal portion, the neck being narrower than a width between the two edges in the body portion;
coining a thinned region in the distal portion, the thinned region spanning the neck;
separating the distal portion along the thinned region after coining; and
forming a longitudinal channel in the elongated shape, the longitudinal channel being aligned with the longitudinal axis, the longitudinal channel being formed out of the major plane to give the elongated shape a U-shaped cross-section in a plane normal to the longitudinal axis at the body portion.

28. The method of claim 27 wherein the neck is hour-glass shaped and the thinned region defines a V-shape in the major plane.

29. The method of claim 27 wherein forming the longitudinal channel in the elongated shape is subsequent to coining the thinned region that spans the neck.

30. The method of claim 29 wherein forming the longitudinal channel in the elongated shape is subsequent to separating the distal portion along the thinned region.

31. A needle, comprising:
a body portion having a longitudinal channel that defines a longitudinal axis;
a distal tip; and
a tapered portion disposed between the distal tip and the body portion, the tapered portion having a U-shaped cross-section in a plane normal to the longitudinal axis, the U-shaped cross-section defining an open top and a closed bottom;
wherein the distal tip has a cut facet that is inclined with respect to the longitudinal axis by an angle in the range 15 degrees to 50 degrees, and
wherein a plane that is parallel to and tangent the cut facet does not intersect any part of the needle except the cut facet.

32. The needle of claim 31 wherein the longitudinal channel is an open longitudinal channel having channel side walls that define a wall height in the range 0.1 mm to 1.5 mm, the wall height being measured normal to the longitudinal axis from the closed bottom.

33. The needle of claim 31 wherein the longitudinal channel is a closed longitudinal channel having a closed hollow cross-section in a plane normal to the longitudinal axis.

34. The needle of claim 31 wherein the distal tip defines a distal tip radius of curvature in a plane that is parallel with the longitudinal axis and tangent to the closed bottom, the distal tip radius of curvature being in the range 12 microns to 125 microns.
